(19) United States
(12) Patent Application Publication
(10) Pub. No.: US 2004/0254441 A1
(43) Pub. Date: Dec. 16, 2004

(54) Method and device for the spatial adjustment of tussue data that are optically acquired at temporal intervals

(76) Inventor: Stephan Schrunder, Berlin (DE)

Correspondence Address: Stephen E Bondura, Dority & Manning, PO Box 1449, Greenville, SC, 29602-1449, US

(21) Appl. No.: 10/474,583

(22) PCT Filed: Apr. 11, 2002
(86) PCT No.: PCT/EP02/04045

(30) Foreign Application Priority Data
Apr. 11, 2001 (DE) 1018314.3

(51) Int. Cl. A61B 5/05

(52) U.S. Cl. 600/407

(57) ABSTRACT

A method for registration of data records of tissue images recorded at temporal intervals includes providing marked structures at fixed locations on the surface of the target tissue to be recorded. At least a first and a second image of the target tissue and first and second images of the marked structures are also recorded at temporal intervals. The position coordinates of the marked structures are computed in three spatial dimensions for the two images. The target tissue images are then registered with a computer based on the position coordinates of the marked structures.

### FIELD OF THE INVENTION

The invention relates to a method as well as to a device for the adjustment, the so-called registration, of tissue data, in particular of an eye, that is optically acquired at temporal intervals.

### BACKGROUND

The registration of tissue data, in particular of images of a tissue surface, requires precise knowledge of the tissue's position at the points in time of tissue data acquisition. The position which changes due to the movements of the object can be followed by means of so-called trackers. Eye movements in particular have been a focus of scientific and clinical interest for some time now, since they provide information on neurological diseases, among other things.

A first method for the measurement of eye movements (the so-called eye tracking) was practiced in the middle of the 19^{th} century. With this method a tested person must look fixedly at the center of a rod-shaped light source. When the light source has been shut off, this tested person perceives an after-image. The tested person was then asked to fix different points on a wall. An assistant attaches a rotatable rod thereon in such manner that it coincides with the after-image. The torsional deviation of the after-image in function of eye direction that can thus be determined can then be analyzed further.

In the 1960's, D. A. Robinson in Baltimore developed the so-called “search coil” method, known in German as “Magnet-Okulographie”. This method allows for the recording of eye movements with high resolution. Hair-thin wires are wound into coils, are cast in silicon and are placed on the eye in the manner of contact lenses. The patient's head is in a frame in which the coils produce a magnetic field. The movement of the eye causes a current to be induced in the coils and this current is measured and represents the corresponding eye movement.

Electro-oculography is another method. The uniformly oriented retinal rods and cones of the eye produce an electric field that is diverted through electrodes affixed near the eye. The oriented field changes with the eye movement.

In infrared oculography, the different reflection and absorption properties of the eye surface are utilized. Irradiation with infrared diodes, for example, determines reflection at the contact surface. The direction of the eye can be deduced from the distribution in the Purkinjian images obtained in this manner. However, this method requires a very detailed model of the different contact surfaces and optical systems in the eye.

Present developments in eye tracking technology are taking place in the area of video oculography. With this method the eye is observed by a camera and the images obtained are evaluated directly online or offline. The evaluation is based in that case on typical image structures. The position of the pupil can be determined for horizontal and vertical excursion while the torsional components are determined by analyzing the iris pattern. Other methods use the transition between iris and sclera, the limbus, as the structure to determine position. Video oculography was able in the last few years to profit mainly from the developments in computer technology, since many computations must be carried out in a short time for the evaluation of the images.

If the eye surface is measured in several measurements at temporal intervals, there is need to adjust, i.e. the recorded images, to cause them to cover each other. This process is also called registering. It is however a common characteristic of the methods mentioned before that their measuring precision is insufficient for highly precise adaptation of eye movements so as to determine the shape of the surface. Tilting movements of the eye are thus difficult to analyze. Although a measuring precision of less than 50 μm is enough to orient individual laser impulses with sufficient precision for a surface treatment of the cornea, deviations of more than 10 μm in the existing methods are unacceptable for repeated measuring of the surface between applications of laser impulses.

### SUMMARY OF THE INVENTION

It is an object of the present invention to further develop a method or a device of the type mentioned initially so that the precision of registration of several tissue data samples acquired by optical means at temporal intervals can be improved. Thus, it is desirable for example to optically detect the position difference of the surface between two points in time occurring unavoidable because of eye movements and to compensate for them so precisely that a subtraction of the measured values at the two points in time may provide information on form change while taking the possibility of a position change into account.

Additional objects and advantages of the invention will be set forth in part in the following description, or may be obvious from the description, or may be learned through practice of the invention.

The advantages of the invention consist in particular in the possibility of determining position changes of the tissue or organ or tissue surface to be measured in three-dimensional space between two detection processes by means of the method or the device according to the invention. For this purpose, the position coordinates of the marked structures that are in fixed position relative to the surface are calculated at least twice at temporal intervals (whereby a suitable electronic memory is used for the first measurement data or the values calculated from them). The essential feature of the invention consists in the fact that the position coordinates are detected or calculated three-dimensionally, i.e. in x, y and z direction. Until now the position coordinates were recorded optically in two dimensions only, i.e. in x and y direction, so that a tilting of the tissue surface could not be detected sufficiently. Furthermore, it is important for the interval between the determination of the first or second position coordinates of the marked structures and the determination of the first or second surface coordinates of tissue data to be sufficiently short so that no positional changes of the tissue and thereby of the marked structures may occur in the interval. The position measurements and appertaining acquisitions of tissue data are therefore preferably carried out simultaneously.

The process makes it possible, in particular, to register images of the tissue surface taken at temporal intervals with each other in order to correct for movements of the tissue between the measurements. In a so-called matching process, the tissue data measured at different times is coordinated by means of a computer in such a manner that movement artifacts can be adjusted. In this manner, form changes of the surface—e.g. due to an induced material paring—that have occurred between the determinations of position can be detected and controlled by forming a differential.

The z coordinate of the marked structures is determined in a preferred embodiment of the invention by projection of a pattern that is observed or detected from an angle that is different from the angle of projection on the marked structures and advantageously on the entire tissue surface. The incident radiation/detection thus takes place at a triangulation angle. This makes it possible for the tissue, and in particular the marked structure, not to be recorded as a plane projection on, e.g. a CCD chip of the camera, but to be calculated by means of a computer as a form in all three dimensions from images, e.g. those of such a camera.

In an alternative embodiment of the invention, measurements of the positions of the marked structures are effected by at least two detection devices, whereby the directions of detection are different from each other. Thereby at least two images are obtained simultaneously of the marked structures and are correlated with each other in order to obtain the position coordinates of the marked structures in the x, y and z direction.

The method according to an embodiment of the invention requires a measuring method for rapid acquisition of tissue data. This method may be, e.g. a so-called stripe projection method by means of which it is possible to measure a surface form of the tissue, or a wave front measuring method (wave front aberration, see e.g. U.S. Pat. No. 5,777,719) or some other measuring method—e.g. based on ultrasound measuring. The tissue data must be available in the three-dimensional data record. The invention then makes it possible to optically detect the difference in position of the surface between two points in time due to unavoidable movements and to compensate for them through computation in such a manner that a subtraction of the measured values at the two points in time supplies information on the change in form while taking into account that a change in position may have occurred.

The method or the device is suited, for example, to detect the change in the eye's position during a laser treatment of the cornea. This is necessary in order to make it possible to plan different surface measurements through which the course of the treatment is to be verified correctly with respect to position. In this manner, the ablation by laser can be detected through the formation of a differential of the surfaces whose positions relative to each other have been corrected. The determination of position thus takes place preferably simultaneously with the surface measurement with the purpose of being able to subtract the individual measured values from each other in order to calculate the actual laser ablation.

Alternatively or in addition, a diagnostic measurement, e.g. the known wave front aberration method can be used as a first tissue acquisition with appertaining position measurement of the marked structures. This method is registered later with a second surface and position measurement, e.g. by means of a pattern projection method as a topography measuring method, shortly before or during the treatment, e.g. in order to actuate and focus or adjust a laser for the treatment according to the treatment need by means of a control unit. In all cases, the advantage exists that the tissue treated or to be treated or its surface is measured so that no error in the determination of position may be made through the conversion of distances between nearby structures in three-dimensional space.

The first and second tissue data acquisitions can therefore be effected according to different measuring principles, whereby these measuring principles may be different from the one or the ones used to measure the positions of the marked structures.

A similar method for eye measurement with diagnostic of the eye defect, providing a treatment model followed by treatment of the cornea, is described in DE 199 50 791 A1, the disclosure of which is herewith incorporated by reference. The “determination of refractive characteristics” mentioned therein is here the acquisition of tissue data. DE 199 50 791 A1 also describes that for the determination of the refractive characteristics cornea topography system, wave front aberration systems and ultrasound systems can be used in order to develop from these in particular a refractive treatment course which is then carried out by means of a control unit with actuation of a treatment laser. Explicit mention is made here of the fact that this is also possible and preferred with the method or device according to the present invention. It is a particular disadvantage with the known method that only two-dimensional images are used, while with the method or device according to the present invention, the coordinates in x, y and z direction are calculated from two-dimensional images.

The first acquisition of tissue data—according to the preceding statements—may be e.g. a diagnosis measurement or a measurement before or during the treatment of the tissue. The second, subsequent tissue data acquisition is then e.g. a measurement before, during or after the tissue treatment, the latter preferably to check the treatment obtained results.

The laser used to measure the tissue data or, in case of measuring the eye, the refractive characteristics and the position of the marked structures for the registration in space of the different photographs, is preferably the same as the laser used for the laser treatment of the surface. In this case, the source of radiation is designed with respect to intensity, duration of pulsations, rate of repetition and wavelength of the excitation radiation for surgical treatment of the biological tissue such as e.g. the ablation of corneal tissue. In this case an intensity attenuator or a ray expander may be indicated between the (at least one) source of radiation and the biological tissue for introduction into and removal from the path of rays of the excitation radiation.

In one embodiment of the method according to the invention, at least three markings or marked structures are necessary in order to define a position change of the surface. For this purpose, a triangle with these markings is formed in space. The perpendicular going through the center of this triangle is altered in case of position change. The two triangles are then registered with each other by means of computed superimposition of the perpendiculars and successive rotation around the perpendicular and displacement along the perpendicular. The displacements in space that are required for this apply in the same manner to the entire surface, so that it too can be registered.

In a preferred embodiment of the invention, the respective tissue data acquisitions and position measurements are effected simultaneously. This ensures that no changes in the position of the tissue occur between position measurement and acquisition of tissue data, so that a precise registration of the data records of the (at least two) tissue data acquisitions can be carried out by registering the position coordinates of the marked structures.

Effecting the tissue data and position measurements by using the previously mentioned pattern projection method and in particular a stripe projection method is advantageous. It has been shown that three-dimensional structures or contours can be measured reliably by using such a method. It is then possible to measure the tissue surface as well as the position coordinates of the marked structures in the area of the tissue surface by projecting a pattern, preferably at the same time and with the same detection device or devices.

Alternatively, other measuring methods possibly based on the wave front aberration method or on ultrasound technology can be used at the same time for the—preferably first—determination structure positions (see above).

An advantageous possibility for the implementation of the above-mentioned projection method (as of that of the stripe projection method) consists in projecting a fluorescence-exciting radiation in the form of a projection pattern on the marked structures to be measured. The means for the production of the irradiation pattern comprises in this case preferably a mask with openings in the form of preferably parallel slits or evenly-spaced holes. The mask is placed between the source of radiation and the tissue. The marked structures are then excited in the area of their surface, preferably for the emission of a fluorescent pattern through suitable selection of the rays' wavelength, whereby this fluorescent pattern matches at least one cutout of the irradiation pattern. The fluorescent pattern can then be detected for the computation of the position of the marked structures and thereby for the registration of the tissue data records of the first and second data acquisition. Tissue data records are recorded in a preferred embodiment of the invention simultaneously and also by means of the stripe projection method. Such a surface measurement is described in DE-PS-198 37 932.3, the disclosed content of which is included herewith. The device described therein for the production of the exciting radiation, the pattern generators and the detection devices are referred to in particular and are included in the present application by reference.

It has been shown to be advantageous if the effective wavelength of the excitation radiation for the production of fluorescent pattern emitted directly from the tissue lies essentially in the ultraviolet range of wavelengths. In measuring the cornea of an eye, UV light has the great advantage that it can render the cornea artificially visible. Visible or infrared light would pass through the cornea so that only images of the iris located in the eye could be obtained. However since the cornea—and not internal eye tissues—is to be corrected by laser ablation for example, no position coordinates of marked structures can be obtained in the area of the cornea with visible or infrared light.

The at least one radiation source is preferably in the form of a laser, preferably in the form of a frequency-multiplied fixed-body laser, excimer laser, gas laser or frequency-multiplied dyestuff laser, or in form of flash lamp, preferably filled with a xenon or deuterium gas mixture.

A UV wavelength range is also indicated when a fluorescent pattern is produced on the basis of a fluorescent dye applied on the or into the tissue from the outside.

In a further development of the method according to the invention, different methods are used advantageously for the determination of the position coordinates. Thus, for example, it is possible to define the real x and y coordinates by scaling the pixel coordinates of the camera and the z coordinate by evaluating the height at which the means of height at which pattern or stripes take their course along the marked structures. The two-dimensional measuring in x and y directions is in this case identical with a photograph according to the state of the art, e.g. by means of a CCD or CMOS camera. The measurement in z direction is realized through perspective observation or detection of the stripes (whereby the change in stripe lightness can be used with the marked structures).

To produce the marked structure, drops of a fluorescent liquid are applied at different points on the tissue. It must be ensured in that case that the liquid remains in stationary position on the tissue, and it is possible to achieve this by pressing the liquid into the outermost tissue surface. During the irradiation of the tissue surface with an exciting pattern the fluorescent material applied is excited to emit florescent radiation, so that its location—corresponding to the x and y coordinates—can be determined by means of a camera through the difference in lightness of the foreign material as compared with the surrounding tissue area. The photograph of the fluorescent pattern emitted on the basis of the irradiated projection pattern which is also visible on the manipulated tissue area provides information on the position of this area in z direction and thereby in particular on a tilting of the tissue. If the tissue is tilted relative to a first position measurement of this tissue area (and relative to a first surface measurement of the tissue surface), the stripes either move closer together on the tissue surface and thereby also on the manipulated tissue area, or are at a greater distance from each other than in the first measurement under perspective observation—i.e. triangulation—by the recording camera. With suitable depth of field of the observing camera, the z component of the manipulated tissue area can be extracted from this information. It goes without saying that these tissue areas must be selected to be large enough so that the pattern can be projected on them in a recognizable manner.

Marked structures of natural origin in particular can be used. For example, veins, pigments and local unevenness etc. can be used for this purpose. Hair on the tissue surface can also be used under certain circumstances, but must then be as immobile as possible. If fluorescent characteristics of the marked structures are used to determine the position coordinates, the number of possible marked structures is reduced. In that case hair is less suitable, for example.

Alternatively or in addition, marked structures are produced artificially. It is also possible to manipulate structures of natural origin. These two methods have the advantage that the user is able to lay out or adapt the marked structures in function of existing requirements. If, for example, a corneal surface is to be measured before and after the removal of a corneal layer, the marked structures can be provided at shorter distances from each other outside the treatment area.

It is especially advantageous to produce or to manipulate marked structures by adding or removing material. In a preferred embodiment of the invention material, in particular fluorescent material, is applied to or pressed on the tissue in this case. It is then able to penetrate into the tissue so that it is not wiped away on the tissue surface.

Alternatively or in addition, at least one body can be placed on the tissue and adhere to the tissue surface with substantial form stability. The body may advantageously contain a fluorescent dye in case of the ablation of corneal tissue.

In a special embodiment, the body is made in the form of a thin film, preferably with a marked surface on which the three-dimensional measurement is based on the side towards a detection device.

Another possibility for the production of the marked structures consists in creasing the tissue or irradiating it with laser pulsations so that the structures thus produced possess fluorescence of a lightness and/or frequency that is different from the untreated tissue and/or can be recognized as three-dimensional tissue change.

If the measured tissue is modified between measurements, e.g. by laser ablation of corneal layers of an eye, the kind of marked structures used or produced are advantageously located outside of the treated segment of the tissue surface. In that case, the marked structures are not influenced by treatment, so that a registration of the tissue data or surface coordinates before and after the treatment is possible thanks to the position coordinates of the marked structures whose position only may have been changed.

In a special embodiment of the invention, a cut is made in a cornea so that a corneal flap can be folded back. A laser impulse can then be directed on the layer beneath for the removal of a corneal layer for the correction of a visual defect. The marked structures are in that case selected or placed preferably in proximity of the edge of the cut. It is possible for the marked structures to be located or to be produced outside this flap.

Alternatively or in addition, a previously defined edge of the cut is itself used as marked structure. It may be advantageous in that case if the cut edge is tilted in such a manner relative to the direction of irradiation and observation that the stripes can be clearly observed on the edge of cut. The utilization of the cut edge as a marked structure has the advantage that no additional structures have to be located or generated. This reduces costs.

In order to ensure constantly that the marked structures can be observed in case of a corneal surface measurement, the eyelids are advantageously held open or opened by suitable holders in such a manner that the lids do not cover or shift the marked structures when eye movements occur.

The device according to the invention can be provided with at least one additional radiation source and/or at least one arrangement for the distribution of the incident or exciting radiation in order to irradiate the biological tissue from at least two directions.

Examples of embodiments are described in greater detail below through the figures.

### BRIEF DESCRIPTION OF THE DRAWINGS

FIG. 1 shows a perspective view of an eye with lifted flap or tag and marked structures outside the edge of the cut;

FIG. 2 shows a top view of the eye of FIG. 1;

FIG. 3 shows a schematic perspective view of an eye with lifted flap and marked structures on the edge of cut;

FIG. 4 shows a top view of the eye of FIG. 3;

FIG. 5 shows a schematic representation of a cornea with edge cut and artificially produced troughs;

FIG. 6 shows a schematic representation of a cornea with edge cut and fluorescent markings applied;

FIG. 7 schematically shows a first device to measure the corneal surface and the position coordinates of marked structures; and

FIG. 8 schematically shows a second measurement device.

### DETAILED DESCRIPTION

Reference will now be made to particular embodiments of the invention, examples of which are illustrated in the drawings. The embodiments are presented by way of explanation of the invention, and are not meant as a limitation of the invention. It is intended that the invention includes modifications and variations to the embodiments described herein.

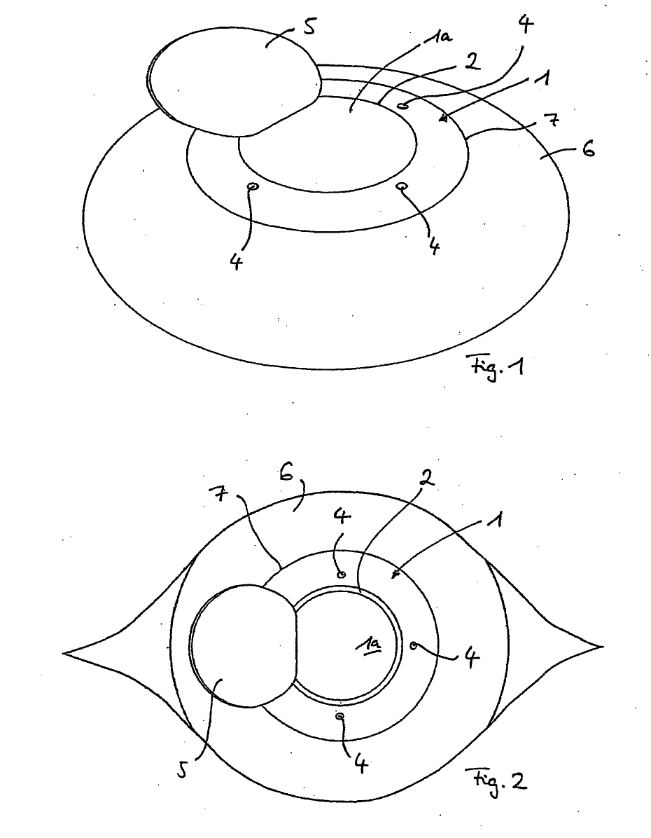

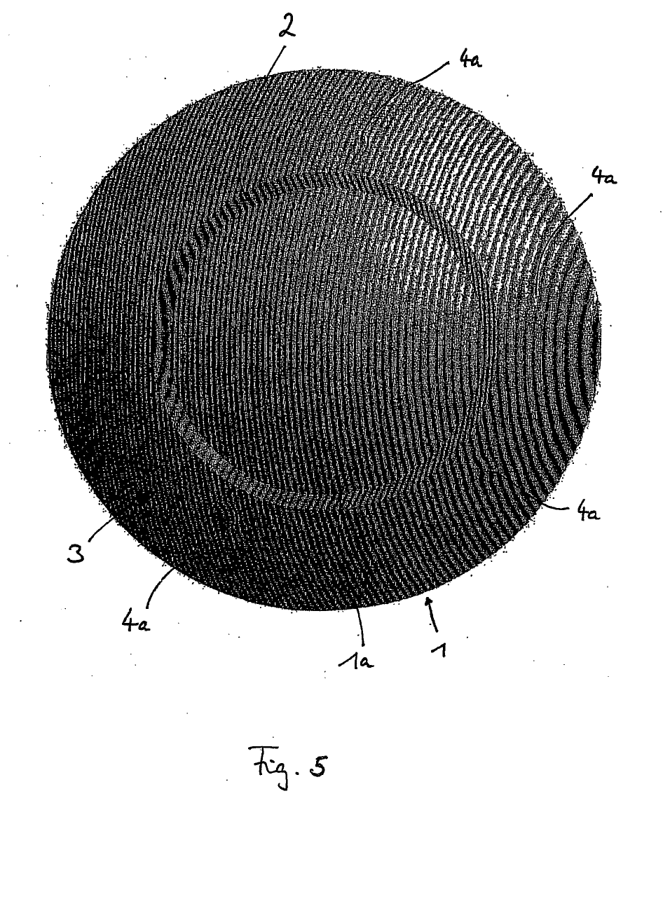

FIGS. 1 and 4 schematically show an eye of which the cornea 1, the sclera 6 and the transition between them, the so-called limbus 7 are shown.

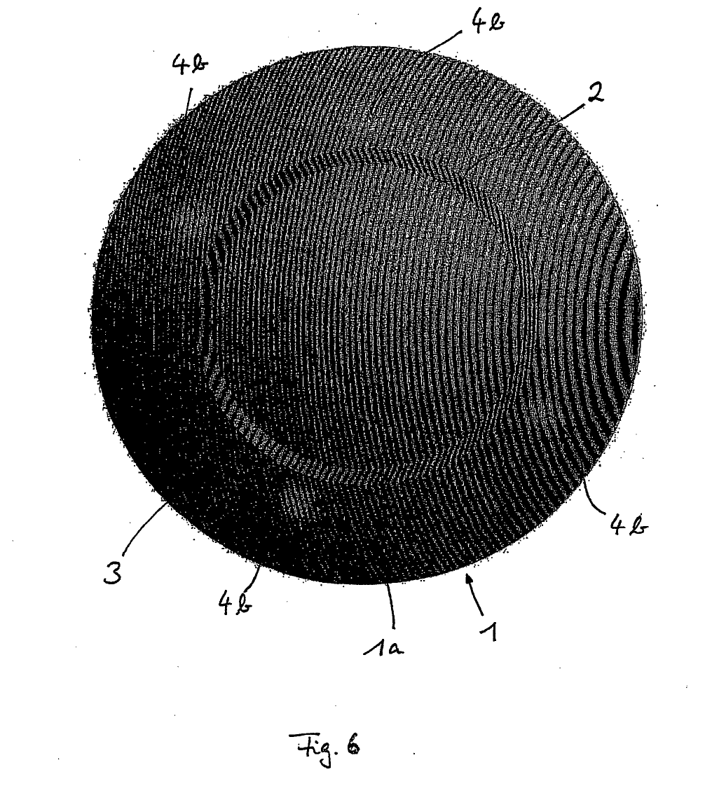

In the examples of embodiments shown, a tag or flap 5 is produced in the center of the corneal surface 1 of the eye by making an edge cut 2 by means of a knife cutting parallel to the surface, a so-called micro-keratom. This flap 5 is then lifted up over a circular surface of up to 10 mm in diameter and folded back for the purpose of further treatment of the bared corneal tissue 1 a by means of a laser. The inner corneal tissue 1a that is bared in this process is then pared by means of a laser emitting preferably pulsations in the ultraviolet (UV) range between 150 and 250 nm. The measuring device by means of which this paring is to be detected consists preferably of a stripe projector by means of which a stripe pattern of UV light is projected on the corneal surface 1 (see FIGS. 5 and 6). The light source in the UV range for the projection is preferably identical with the laser for the treatment of the corneal area 1a by ablation. The tissue or cornea 1 emits fluorescent light in the irradiated areas in the form of the fluorescent pattern 3 of the projection pattern. This fluorescent light is photographed by a camera from a detection direction that is different from the incident irradiation direction and is evaluated, e.g. by means of a PC. The surface form of the cornea 1 is then available as a data record with three-dimensional data. Further details concerning this method, including different designs of the required irradiation and detection devices are contained in DE-PS-198 37 932.3

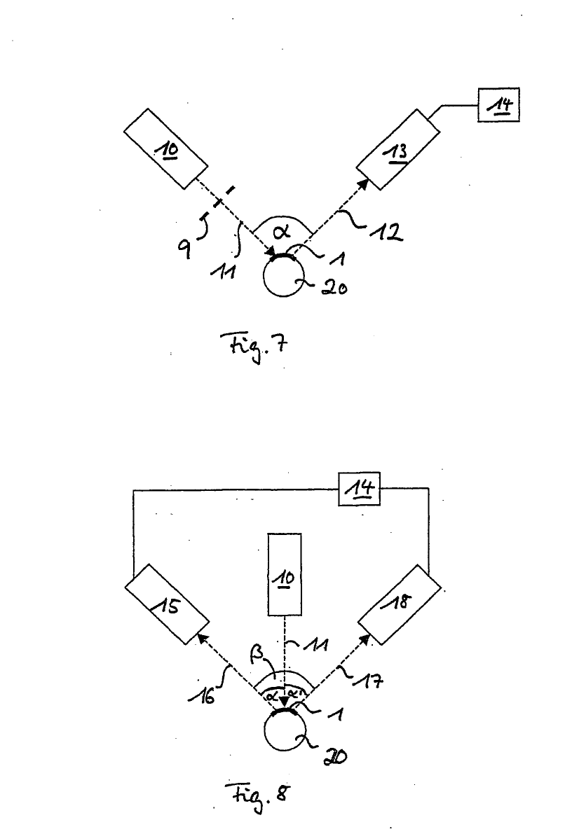

FIG. 7 schematically shows the device described above. The source of radiation or laser 10 radiates an irradiation pattern produced by a pattern generating device 9 (e.g. a slit mask) on the cornea 1 of an eye 20 from a direction 11. The fluorescent pattern produced is detected from direction 12, forming an angle alpha α greater than 0° with direction 11, by the detection device 13 and is evaluated by a computer. The result contains the surface form of the cornea 1. For the sake of simplification, FIG. 7 (as also FIG. 8) focusing and possible enlarging lens systems, ray deflection devices, filters, intensity attenuators, partially transparent mirrors etc. are not shown.

Since the paring of the corneal surface 1a takes place in several steps (e.g. by means of controls, not shown, of laser 10 by computer 14 on the basis of a calculated refractive treatment plan), it is most desirable to verify the success of each step. For this purpose, the surface to be treated and its surrounding surface are photographed after every treatment step. In order to register these photographs in correct position, intermediate movements of the eye must be taken into account. This process is called adjustment or registration of the photographs. According to the invention, marked structures 4 whose position coordinates in three-dimensional space are determined at suitable points in time by photographing two-dimensional images of the marked structures 4—with corresponding intermediate storage of the images or equivalent data—followed by computation of the x, y and z coordinates, are used for the registration of the surface photographs or measurements. Thanks to the detection, according to the invention, of the position of the marked structures in three-dimensional space, tilting of the corneal surface in particular which cannot be detected with conventional measurements can be taken into account.

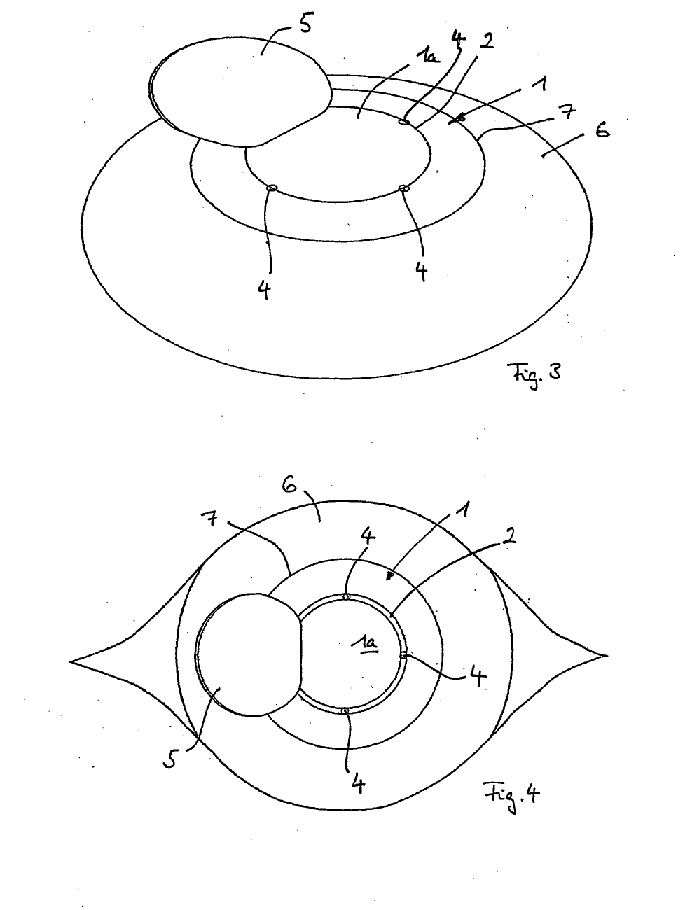

In the embodiments of FIGS. 1 and 2, three markings or marked structures 4 with small diameter are artificially affixed on the corneal surface outside the bared corneal tissue 1a. In the embodiment of FIGS. 3 and 4 three marked structures 4 are also provided, but this time on the edge cut 2. This placement choice has the advantage that the structures 4 are located very close to the treatment surface without themselves being affected by the treatment.

Examples on how the marked structures can be produced and how the registration can be carried out are explained below through FIGS. 5 and 6 in which only the cornea 1 without tag or flap 5 is shown for the sake of clarity.

FIG. 5 schematically shows how the surface 1 of the outermost corneal layer (the self-regenerating epithelium) was treated with individual laser impulses of small diameter so that local markings were produced—designated marked structures in the sense of the invention. These structures are recognizable as troughs 4a with small surfaces and few micrometers deep with a diameter of e.g. 500 μm in the three-dimensional measurement. The troughs 4a are applied before the actual surgery in the area outside the previously cut flap in the self-regenerating epithelium by the laser used for the treatment. Alternatively the troughs 4a are produced on the edge cut 2 of the flap 5 which is in greater proximity of the laser treatment location and whose three-dimensional position can therefore be measured more reliably (see FIGS. 3 and 4).

The position coordinates of the marked structures formed in the corneal surface 1 in the form of troughs 4a are photographed by means of a camera. In FIG. 7 this camera is the detection device 13. The x and y coordinates of the troughs 4a can be determined in the corresponding photographs in the usual manner by determination of lightness. The z coordinates are determined by the course of the fluorescent pattern 3 that corresponds to the projected stripe pattern emitted from the trough structure. The form and the depth of each trough 4a influences the course of the pattern observed by the camera from which the depth or position of the trough 4a in question can be determined in all three spatial directions and in particular in the z direction, preferably by means of the computer 14 for the embodiment shown in FIG. 7.

The determination of the position coordinates of the troughs 4a by measuring their position coordinates at two different points in time allows for the three-dimensional registration of the surface coordinates from two photographs of the corneal surface 1. For this, the position of the surface 1 must not change between the first (or second) position measurement and the first (or second) surface measurement. It is therefore indicated to carry out the corresponding position and surface measurements simultaneously and by the same method, preferably as described, by means of the pattern or stripe projection method by projecting the (stripe) pattern on the corneal surface 1 to be measured as well as on the troughs 4a (see also FIG. 7). The above-mentioned registration is carried out by means of a computer, in case of the embodiment of FIG. 7 preferably the computer 14 which serves in this case also as registration device 14.

Structures can also be produced by means of laser impulses that are not necessarily in the form of troughs 4a, but possess fluorescence with lightness or color that is different from those of the untreated epithelium, so that a three-dimensional determination of the positions of these structures is possible through these characteristics.

In the embodiment shown in FIG. 6, the marked structures are in the form of clearly recognizable fluorescent markings 4b. For this, local and stationary markings 4b are applied e.g. by means of eye-compatible drops (e.g. fluorescine) on a small area of the cornea. The markings 4b can also be applied with a special cornea marker such as is already being used today in modified form for the recognition of the lifted corneal flap. In this process, thin tips are pressed on a kind of stamp pad containing the marking color. The marker is then pressed on the suitable location on the cornea under microscopic observation. The dye then penetrates the outer epithelium layer and is deposited therein. If necessary, excess dye that would render the marking 4b too large for reliable recognition can be removed by means of a rinsing solution. It is however preferred to achieve precise dosage of the dye quantity, and this can be achieved through suitable design of the tips of the marker. Thus the tips should resemble in configuration very thin tweezers by means of which a certain quantity of liquid can be aspired on the basis of capillary force. This liquid is released at contact with the cornea into the depression caused by the pressure of the tip. The marking 4b then has preferably a diameter of less than 500 μm. Different embodiments of the marker have tips at different distances from each other that can be selected in function of the diameter of the previously produced flap.

The position coordinates of the fluorescent markings 4b are detected in analog manner to the method described through FIG. 5, e.g. by means of the arrangement according to FIG. 7. The x and y coordinates are calculated in the know manner from the respective photographs and the z coordinates, again from the course of stripes of the fluorescent pattern 3. If a detection device with a CCD or CMOS chip is used, the course of the stripes over the markings should be clearly recognizable thanks to suitable dosage of the fluorescent dye and should advantageously not be too light so as to avoid overexposure of the chip.

In the two cases shown in FIGS. 5 and 6, the stripe width and the distance between stripes are advantageously smaller than the marked structures 4a, 4b themselves for the sake of sufficient resolution. The stripe width and the interval between stripes are e.g. 50 μm, so that the corneal surface 1 and the marked structures 4a, 4b can be measured with a resolution of a few micrometers. In principle, a plurality of suitable patterns can be used, e.g. grids whose intersection points can be used for evaluation, hole patterns, patterns consisting of several concentric circular lines with lines extending radially from the center and at the same angular distance from each other, moire patterns consisting of two line patterns, or other geometrical patterns.

Instead of the structures of FIG. 5 in form of troughs 4a, or the structures 4b of FIG. 6, marked with fluorescent material, the three-dimensional form of the edge cut itself can be used alternatively if it can be differentiated sufficiently in order to make position measuring possible. This is the case according to FIGS. 5 and 6, since the edge cut 2 can be observed very well and the stripes stand out well against it. For a determination of position, the freely accessible area of the edge cut 2 must be detected completely in three-dimensional space and must then be recognized again through suitable algorithms that detect, e.g. the course of the curvature of the edge 2 in space.

Another embodiment not shown of a marked structure consists of one or several bodies installed preferably on the cornea outside the flap. These are preferably bodies of a material with good adhesion to the corneal surface 1 which is moist to begin with while the body itself does not change permanently in form due to the absorption of moisture from the cornea. A thin, ring-shaped film adhering to the corneal surface 1 outside the flap can be used in this case. On its side towards the camera, this film is provided with a marked surface on its side towards the camera that can be measured in space in suitable manner by means of the method used. On its side towards the cornea, the film is advantageously made so that it may lie as tightly as possible against the cornea so that it may not be displaced by eye movements during surgery. This risk is avoided mainly if the film is relatively thin, e.g. with a thickness of 20-50 μm. Thorough rinsing with a liquid is used to easily remove the film after surgery.

With marked structures of the type mentioned above in particular, it is advisable to detect the corneal surface and in particular the marked structures by means of two detection devices 15, 18 from at least two directions 16, 17 forming an angle β and to adjust them by means of a computer 14 as shown in FIG. 8. The projection of a pattern is not required for this, although it is possible. The radiation source 10 shown in FIG. 8 where the incident angle of irradiation 11 forms an angle α with the detection direction 16 as well as an angle α′ with the detection direction 17 serves in this case merely to illuminate the cornea 1. The measurement of the optical imaging system of the eye or of the different refractive eye segments can be effected e.g. by means of a wave front aberration system not shown here, preferably in every case at the points in time of the respectively appertaining position measurements of the marked structures.

It is however also possible to project a pattern (or even several patterns, possibly from different directions) on the cornea 1, similarly to FIG. 7, whereby one or both detection devices 15, 18 are used to measure the surface by means of the previously described fluorescent pattern detection. The two detection devices 15, 18 can also evaluate the fluorescent signals emitted by the structures—in addition to detecting the position coordinates of the marked structures thanks to their respective placement at angles—in order to have the position coordinates computed by the computer 14. The registration of the surface measurements can advantageously also be effected by the computer 14.

It should be mentioned that detection by means of at least two detection devices according to FIG. 8, detecting the tissue surface and in particular the marked structures from at least two directions, is not limited to the detection of externally applied structures such as the film described earlier.

All embodiments have in common that the marked structures 4, 4a, 4b are advantageously such that they can be reliably detected with stripe projection or general pattern projection, and their position in space can be determined reliably. Furthermore the markings should advantageously remain unchanged during the treatment, so that they are fixed on the edge of or outside the micro-keratom cut. Otherwise such a change in position of the structures 4, 4a, 4b relative to the tissue would be erroneously interpreted as a tilting of the surface.

With the schematically outer corneal segments of FIGS. 5 and 6, four marked structures 4a, 4b have been provided or generated in either case. Based on these four structures 4a, 4b, it is possible to detect a position change of the corneal surface. The four structures 4a, 4b comprise a square in space whose perpendicular going through the center changes place in case of a position change. The perpendiculars are superimposed on each other by means of the computer, are successively rotated around the perpendicular and are then displaced along the perpendicular until the two squares coincide. The movements in three-dimensional space carried out in this process apply also to the entire surface, so that the surface coordinates of the (at least two) surface measurements can also be converted into each other.

As can be seen from what precedes, the spatial coordinates of the marked structures 4, 4a, 4b on the cornea or on the tissue surface, of which it is known that they represent the change in position and do not themselves change position between measurements relative to the surface itself, are necessary for highly exact detection of a position change in three-dimensional space. These structures 4, 4a, 4b are preferably provided or selected before the measurements in the area of the surface that remained unchanged. It is essential in that case that the structures will be detected not only in two dimensional but also in three-dimensional space with their space coordinates. This provides the possibility that a three-dimensional structure recognition going beyond the conventional, two-dimensional pattern recognition, by means of a position change of the tissue surface can then be achieved.

In other words it has been shown that the two-dimensional imaging detection of the tissue surface of the state of the art is suitable for a highly precise determination of position or position change over time only if the type of position change of the tissue surface would be known a priori. This could be e.g. the knowledge of the position and orientation of axes of rotation or points of rotations, or the certainty that only a lateral displacement occurs. However this is not the case with moving tissues. For highly precise determination of the position change in three-dimensional space, the coordinates of the marked structure in the area of the surface are therefore used according to the invention in all three spatial directions of which it is known that they represent the position of the tissue surface and do not themselves change position relative to the surface between measurements.

It should be apparent to those skilled in the art that various modifications and variations can be made to the embodiments of the invention described herein without departing from the scope and spirit of the invention as set forth in the appended claims and their equivalents.

1-33: (Cancelled)

34. A method for registration of data records of tissue images recorded at temporal intervals, comprising:
providing marked structures at fixed locations on the surface of the target tissue to be recorded;
recording at least a first and a second image of the target tissue and first and second images of the marked structures at a temporal interval;
computing with a computer the position coordinates of the marked structures in all three spatial dimensions for the two images; and
registering the target tissue images with the computer based on the position coordinates of the marked structures.

35. The method as in claim 34, wherein the target tissue is tissue of an eye.

36. The method as in claim 34, wherein the target tissue and the marked structures are recorded in the same images.

37. The method as in claim 34, wherein the target tissue and the marked structures are recorded in different respective images.

38. The method as in claim 34, wherein the marked structures are fixed in position relative to the surface of the tissue such that there is essentially no change of position of the marked structures relative to the target tissue between the recordings of the target tissue.

39. The method as in claim 34, wherein the target tissue is recorded with the marked structures in first and second photographs.

40. The method as in claim 34, further comprising projecting a pattern on the marked structures at an angle of incidence, and detecting reflected radiation from the projected pattern at an angle of detection, wherein an angle α is defined between the angle of incidence and the angle of detection that is greater than 0°.

41. The registration method as in claim 40, wherein the pattern is also projected onto and detected from the target tissue for tissue data acquisition associated with the first and second images.

42. The registration method as in claim 41, wherein the pattern is projected in the form of a stimulating radiation producing a detectable fluorescent pattern from the target tissue and marked structures.

43. The registration method as in claim 42, wherein the stimulating radiation is in the ultraviolet range.

44. The registration method as in claim 40, wherein the x and y coordinates of the marked structures are computed as a function of lightness measurements from the images of the marked structures, and the y coordinate of the marked structures is computed as a function of pattern measurement.

45. The registration method as in claim 34, wherein the first and second images of the target tissue are recorded by means of a measuring principle that is different from a measuring principle used to obtain respective images of the marked structures.

46. The registration method as in claim 45, wherein the first and second images of the target tissue are recorded by a wave front measuring technique.

47. The registration method as in claim 34, wherein the images of the marked structures are recorded by detection of the marked structures from at least two different directions of observation.

48. The registration method as in claim 34, wherein the first images of the target tissue and the marked structures are taken at the same time, and the second images of the target tissue and marked structures are taken at the same time.

49. The registration method as in claim 34, wherein data acquired from the registered images of the target tissue is used in a subsequent corrective procedure conducted on the target tissue.

50. The registration method as in claim 49, wherein the target tissue is an eye, and the corrective procedure is a refractive course of treatment of the eye cornea.

51. The registration method as in claim 34, wherein the marked structures are natural inherent characteristics of the target tissue.

52. The registration method as in claim 34, wherein the marked structures are defined by adding or removing a material from the target tissue.

53. The registration method as in claim 34, wherein the marked structures are defined by laser impulses and possess fluorescence of different lightness or frequency than tissue untreated by the laser impulses.

54. The registration method as in claim 34, wherein the marked structures are placed beyond regions of the target tissue to be treated in a subsequent laser ablation procedure.

55. The registration method as in claim 34, wherein the target tissue is a cornea of an eye and a cut is made in the eye to define a corneal flap that is lifted in a subsequent treatment procedure, the marked structures defined in an area at an edge of the cut.

56. The registration method as in claim 55, wherein the marked structures are defined beyond the corneal flap.

57. The registration method as in claim 55, wherein the cut edge defines the marked structures.

58. The registration method as in claim 55, further comprising holding the lid of the eye open with a holding device to prevent the eye lid from displacing the marked structures.

59. The registration method as in claim 34, wherein the target tissue is a cornea and is to be treated with a laser in a subsequent laser ablation procedure, wherein the same laser is used for the laser ablation procedure and for recording the images of the marked structures.

60. A system for registration of data records of tissue images recorded at temporal intervals, comprising:
a radiation source, and means for production of a radiation pattern from said radiation source onto target tissue at an angle of incidence, the target tissue having marked structures associated therewith;
a detection device positioned to detect and generate first and second images at temporal intervals of reflected radiation from the projected pattern at an angle of detection, wherein an angle α is defined between the angle of incidence and the angle of detection that is greater than 0°;
a computer in communication with said detection device, said computer configured to receive and compute from the first and second images position coordinates of the marked structures, and to register the tissue images taken at a temporal interval with the computed position coordinates of the marked structures.

61. The system as in claim 60, wherein said radiation source produces radiation in the ultraviolet (UV) range.

62. The system as in claim 60, wherein said radiation source comprises a laser.

63. The system as in claim 62, wherein said laser is one of frequency-multiplied solid body laser, excimer laser, gas laser, frequency-multiplied dye laser, or flash bulb filled with a xenon or deuterium gas mixture.

64. The system as in claim 60, wherein said radiation pattern production means comprises a mask having opening defining the pattern.

65. The system as in claim 64, wherein said openings are arranged as a pattern of parallel slits defined at spaced regular intervals.

66. The system as in claim 60, wherein said detection device comprises one of a CCD or CMOS camera.

67. The system as in claim 60, wherein said detection device comprises at least two cameras disposed to detect the reflected radiation at different angles of detection.

68. The system as in claim 60, wherein said detection device comprises at least two detectors disposed to detect the reflected radiation at different angles of detection, and said computer computes the positional coordinates of the marked structures in the x, y, and z dimensions and registers the images of the tissue in the x, y, and z dimensions.

69. The system as in claim 60, wherein said radiation source is suitable for surgical treatment of biological tissue.

70. The system as in claim 69, wherein the target tissue is an eye cornea, and said radiation source is configured with a control unit for performing a subsequent corneal ablation.

71. The system as in claim 70, further comprising a data acquisition device configured to derive and evaluate data from the tissue images for use in the corneal ablation procedure.

72. The system as in claim 71, further comprising wave front aberration means for generating the tissue images at temporal intervals.

73. The system as in claim 71, wherein the control unit is interfaced with said data acquisition device such that repeated registrations of the tissue images are generated during the course of the corneal ablation procedure.
